(19) United States
(12) Patent Application Publication
(10) Pub. No.: US 2022/0258526 A1
(43) Pub. Date: Aug. 18, 2022

(54) GLITTER AND USE THEREOF

(71) Applicant: Sigmund Lindner GmbH, Warmensteinach (DE)

(72) Inventor: Erwin Pschierer, Kastl (DE)

(73) Assignee: Sigmund Lindner GmbH, Warmensteinach (DE)

(21) Appl. No.: 17/674,402

(22) Filed: Feb. 17, 2022

(30) Foreign Application Priority Data
Feb. 17, 2021 (EP) 21157546.9

(51) Int. Cl. B44C 1/10; B44F 1/02; B44C 1/14

(52) U.S. Cl. CPC B44C 1/105; B44C 1/14; B44F 1/02

(57) ABSTRACT

The present invention relates to a glitter comprising a foil coated with metal pigments, more particularly aluminium pigments, to a method for producing it, to the use thereof in cosmetic products, coating materials such as paints, varnishes and printing inks, for example, and in adhesives and modelling and/or moulding compositions, and also to cosmetic products, coating materials and adhesives, and modelling and/or moulding compositions which comprise the glitter.

The present invention relates to a glitter comprising a foil coated with metal pigments, more particularly aluminium pigments, to a method for producing it, to the use thereof in cosmetic products, coating materials such as paints, varnishes and printing inks, for example, and in adhesives and modelling and/or moulding compositions, and also to cosmetic products, coating materials and adhesives, and modelling and/or moulding compositions which comprise the glitter.

### BACKGROUND

Glitters are much employed for producing a sparkling surface effect, and have a variety of uses, particularly in cosmetic articles, paints, varnishes, printing inks, adhesives or play materials, such as modelling and moulding compositions or finger paints, for example. For example, molding compounds comprising glitters are known from EP 0803 549 A2 of EP 1 566 419 A1.

Such glitters are typically produced using foils or films of plastic which by means of a cutting operation are cut into individual particles of comparably small size. At present it is primarily films of polyethylene terephthalate that are used for producing glitter. To a lesser extent, films made of biodegradable films such as polylactose, cellulose acetate or cellulose, for example, are used for producing glitter. An illustrative method for producing such glitters is disclosed in DE 102010001971 A1. Disclosed therein are glitters which are coated from all sides. A further method for coating glitter can be taken from EP 2 918 630 A1.

Foils used for the production of glitter are mostly coated with metals (typically aluminium), which produces a reflective surface and allows a pronounced sparkle effect to be produced. The metal is typically deposited on the foils in a vacuum process. This process is known and is employed commercially for a variety of foils. EP 0769371 A2 discloses the production of metallized polyolefin foil. Metallized polyester foils are cited in DE 69305336 T2. DE 2856510 A1 discloses a method for producing a specular gloss metallization on a substrate.

From an environmental standpoint it is desirable to produce glitters from biodegradable materials. Known biodegradable foil materials, such as polylactose, cellulose acetate and cellulose, for example, are available commercially with aluminium layers deposited by vacuum processes onto the surface.

Cellulose and cellulose derivatives (e.g. cellulose acetate) are known to have high degradation rates under various conditions (e.g. composting, freshwater or saltwater). Foils based on cellulose or cellulose derivatives therefore offer very good prospects for the production of biodegradable glitters.

There are known metallized foils which consist partly of cellulose and have on their surface an aluminium layer applied by means of a vacuum process. For example, these foils are marketed under the brand names Natureflex NM and Natureflex NKM (from Futamura Chemical Co., Ltd.). Glitters marketed under the SiLiglam PURE BIO SPARKLE designation (Sigmund Lindner GmbH) include regenerated cellulose coated with aluminium by means of vacuum processes. EP 3552666 A1 teaches the production of glitter based on cellulose acetate foils. EP 3666830 A1 discloses the production of glitter on the basis of vacuum-metallized cellulose foil.

Numerous cosmetic formulations and also many paints, varnishes, other coating materials, adhesives, or modelling and moulding compositions contain water. From an environmental standpoint it is desirable to use biodegradable glitters in water-based formulations of these kinds. From practice it is known that glitters produced from vacuum-metallized foil on the basis of cellulose have a low dimensional stability in contact with water, and undergo deformation or develop a wavy surface. Moreover, the shelf life in aqueous formulations is limited, as aluminium in an aqueous environment may pass into solution in accordance with the following equation:

2Al+6H₂O→2Al³⁺(aq)+6OH⁻(aq)+3H₂

There are also similar reactions for other metals which may be used in coated glitters.

There is a need for glitters which are biodegradable and remain dimensionally stable in water-based formulations. A further desire is for a very long storage stability in water-based formulations.

The object on which the invention is based, therefore, is that of providing biodegradable glitters which exhibit enhanced dimensional stability and storage stability in water-based formulations.

### SUMMARY

It has emerged, surprisingly, that glitters coated with metal pigments, more particularly aluminium pigments, contrast with vacuum-metallized glitters by having a significantly longer storage stability and being dimensionally stable in water-based formulations.

Biodegradable glitter has to date been produced primarily using foils with a metallization applied by means of vacuum vapour deposition. It has emerged, surprisingly, that the object of the invention, namely biodegradable glitters with enhanced dimensional stability and storage stability in water-based formulations, can be achieved by producing glitter using foils based on biodegradable polymers, the foil being coated with metal pigments, more particularly aluminium pigments, and not coated by means of vacuum metallization.

The present invention therefore relates to a coated glitter comprising a foil which has a coating comprising metal pigments, more particularly aluminium pigments, where the foil comprises cellulose and/or a derivative thereof.

The invention further relates to the use of the coated glitter in cosmetic formulations, coating materials, adhesives, and modelling and/or moulding compositions, and also to cosmetic formulations, coating materials, adhesives, and modelling and/or moulding compositions comprising the coated glitter.

Further disclosed is a method for producing a coated glitter, comprising: providing a foil, where the foil comprises cellulose and/or a derivative thereof; producing a coating comprising metal pigments, more particularly aluminium pigments, on at least one side of the foil; and producing the coated glitter; or providing a foil, where the foil comprises cellulose and/or a derivative thereof; cutting the foil into suitably sized uncoated glitter particles; and coating the uncoated glitter particles with metal pigments, more particularly aluminium pigments.

Further aspects of the present invention are apparent from the dependent claims and from the detailed description.

### BRIEF DESCRIPTION OF THE FIGURES

The appended drawings are intended to illustrate embodiments of the present invention and to impart a further understanding thereof. In connection with the description, they serve to explain concepts and principles of the invention. Other embodiments and many of the stated advantages are apparent in relation to the drawings. The elements in the drawings are not necessarily shown to scale relative to one another. Elements, features and components which are the same, the same in function and the same in effect are each provided with the same reference symbols in the figures of the drawings, unless otherwise stated.

FIGS. 1 to 5 show illustrative glitters of the invention schematically.

### DETAILED DESCRIPTION

### Definitions

Unless otherwise defined, technical and scientific expressions that are used herein have the same meaning as commonly understood by a person skilled in the field of art of the invention.

Quantitative details in the context of the present invention refer to wt %, unless otherwise indicated or evident from the context.

Glitters are small particles having a wide variety of different shapes. In particular they have a size, for example a maximum diameter in a principal extent direction of the particle, of 0.02 mm to 7.0 mm, preferably 0.050 mm to 6.0 mm, for example 0.06 mm to 2.0 mm, e.g. 0.1 mm to 0.5 mm, for example from 100 μm to 400 μm. In terms of the shape, the glitter particles are not subject to particular restriction, and may take the form, for example, of platelets, needles, cuboids, etc., or may have been punched or cut into defined shapes, examples being hexagons, squares, circles, ovals, stars, etc. According to certain embodiments, the glitters have a flat configuration, in the form, for example, of platelets with any of a very wide variety of different shapes, including, for example, hexagonal, rectangular, square, star-shaped, round, oval, etc., and the thickness of the platelets in this case may preferably be between 4 μm and 50 μm, for example between 5 μm and 45 μm, e.g. between 10 μm and 35 μm, illustratively between 14 μm and 23 μm, and/or the size, for example a maximum diameter in a principal extent direction of the glitters, may be from 0.02 mm to 7.0 mm, preferably 0.050 mm to 6.0 mm, for example 0.06 mm to 2.0 mm, e.g. 0.1 mm to 0.5 mm. According to certain embodiments, glitters take the form of squares or rectangles having a maximum length of 0.02 mm to 7.0 mm, preferably 0.050 mm to 6.0 mm, and/or a thickness of 10.0 to 50.0 μm.

In a first aspect the present invention relates to a coated glitter comprising a foil which has a coating comprising metal pigments, more particularly aluminium pigments, wherein the foil comprises cellulose and/or a derivative thereof.

The foil comprises cellulose and/or a derivative thereof. These are biodegradable polymers which have an enhanced dimensional stability and storage stability in water-based formulations in conjunction with the coating comprising metal pigments. In accordance with the invention there are no particular restrictions on the cellulose and derivatives thereof. Derivatives of cellulose are, for example, esters of cellulose, like cellulose acetate, cellulose propionate, cellulose acetate propionate, cellulose butyrate, and/or cellulose acetate butyrate. In addition, also regenerated cellulose is considered to be a cellulose derivative within the scope of the invention. The foil may accordingly comprise, for example, cellulose, regenerated cellulose or cellulose hydrate (e.g. cellophane, hydrated cellulose), cellulose acetate, glassine, or a mixture of two or more thereof, as cellulose and derivative thereof. According to certain embodiments, the foil comprises cellulose. According to certain embodiments, the foil comprises cellulose acetate.

This, however, is not to exclude the foil comprising further constituents, for example at least one further polymer such as a cellulose derivative as well as cellulose, as indicated above, but also, for example, polylactic acid (PLA), polyvinyl alcohol, polyhydroxyalkanoate (PHA), and/or polybutylene adipate-terephthalate (BTA copolyester).

According to certain embodiments, the foil comprises at least 60 wt % of cellulose and/or a derivative thereof, based on the weight of the foil, preferably at least 70 wt % of cellulose and/or a derivative thereof, based on the weight of the foil, more preferably at least 80 wt % of cellulose and/or a derivative thereof, based on the weight of the foil, more preferably still 90 wt % of cellulose and/or a derivative thereof, based on the weight of the foil. According to certain embodiments, the foil consists substantially of cellulose and/or a derivative thereof, thus comprising at least 95 wt %, 98 wt %, 99 wt %, 99.5 wt % or even 99.9 wt % of cellulose and/or a derivative thereof, based on the weight of the foil, or even consists of cellulose and/or a derivative thereof.

According to certain embodiments, the foil comprises at least 60 wt % of cellulose, based on the weight of the foil, preferably at least 70 wt % of cellulose, based on the weight of the foil, more preferably at least 80 wt % of cellulose, based on the weight of the foil, more preferably still 90 wt % of cellulose, based on the weight of the foil. According to certain embodiments, the foil consists substantially of cellulose, thus comprising at least 95 wt %, 98 wt %, 99 wt %, 99.5 wt % or even 99.9 wt % of cellulose, based on the weight of the foil, or even consists of cellulose.

According to certain embodiments, the foil comprises at least 60 wt % of cellulose acetate, based on the weight of the foil, preferably at least 70 wt % of cellulose acetate, based on the weight of the foil, more preferably at least 80 wt % of cellulose acetate, based on the weight of the foil, more preferably still 90 wt % of cellulose acetate, based on the weight of the foil. According to certain embodiments, the foil consists substantially of cellulose acetate, thus comprising at least 95 wt %, 98 wt %, 99 wt %, 99.5 wt % or even 99.9 wt % of cellulose acetate, based on the weight of the foil, or even consists of cellulose acetate.

In addition, however, there may also be other additives present in the foil, with no particular restriction on such additives. The foil may also contain no additives. Alternatively or additionally, according to certain embodiments, the foil may comprise additives which are able to act, for example, as anti-blocking and/or lubricity assistants. There is no particular restriction on these anti-blocking and/or lubricity assistants and they may, for example, comprise amorphous silicas, crystalline silicas, fatty acid amides and/or talc. The amount in which such anti-blocking and/or lubricity assistants are added may, according to certain embodiments, be 0-2.5 wt %, preferably 0-1.5 wt %, more preferably 0-1 wt %, based on the weight of the foil.

Alternatively or additionally the foil, according to certain embodiments, may comprise pigments and/or fillers, which may influence, for example, the colour or the mechanical properties. There is no particular restriction on these pigments and/or fillers, which may comprise, for example, calcium carbonate, barium sulfate, inorganic colour pigments, organic colour pigments and/or mica. There is no particular restriction on the mass fraction which in this case may preferably be less than 40 wt %, more preferably less than 30 wt %, more preferably still less than 20 wt %, and especially preferably less than 10 wt %, for example less than 5 wt %, or indeed less than 1 wt %, based on the weight of the foil. The foil may also contain no pigments and/or fillers. Alternatively or additionally, according to certain embodiments, the foil may comprise plasticizers and/or humectants. These plasticizers and/or humectants are not subject to particular restriction and may comprise, for example, glycerol, urea and/or triacetin. The amount in which such plasticizers and/or humectants are added is not subject to particular restriction and may according to certain embodiments be 0-30 wt %, preferably 0-15 wt %, more preferably 0-10 wt %, based on the weight of the foil.

The foil has a coating which comprises metal pigments, more particularly aluminium pigments. Although aluminium pigments are preferred in the coating, it is possible alternatively or additionally for different pigments to be present. Examples of suitable metal pigments include aluminium pigments, copper pigments, silver pigments, and/or gold pigments, and/or alloys of the stated metals.

There are no particular restrictions here on the application of the coating, and the coating may be present on one side, on two opposite sides, on the entire foil or else in another way, including, for example, partially. According to certain embodiments, the coating is present at least on one side of the foil, for example on a top side as surface. Application may take place in any way, for example from the gas phase and/or liquid phase—in which case the liquid phase may also comprise solids, for example from suspension, etc. According to certain embodiments, coating takes place from the liquid phase using—for example—a suspension, a dispersion, etc.

According to certain embodiments, the metal pigments in accordance with the invention are applied with a coating material. Correspondingly, a coating in the coated glitter of the invention may also comprise at least one coating material, which is not subject to particular restriction.

There is no particular restriction on the coating material which contains the metal pigments, more particularly aluminium pigments, and it may comprise typical coating materials for cellulose-based materials and/or for glitters. According to certain embodiments, the coating material may be a material based on cellulose or modified cellulose, preferably based on cellulose nitrate, cellulose acetate butyrate, cellulose acetate propionate and/or cellulose acetate, and/or based on polyvinyl alcohol and/or shellack and/or polyurethane and/or on polyvinylpyrrolidone, and/or based on natural resins, preferably dammar and/or kopal and/or sandarak gum and/or gum Arabic and/or based on lignin and/or on zein, or may be produced therefrom. According to certain embodiments, the stated binders may have a fraction of 10-99.9 wt %, based on the weight of the coating material. In addition, according to certain embodiments, the coating material may include pigments and/or fillers, which may influence, for example, the colour or the technical properties. There is no particular restriction on these pigments and/or fillers as additives, and they may, for example, comprise calcium carbonate, barium sulfate, inorganic colour pigments, organic colour pigments and/or mica. The mass fraction here may be preferably less than 40 wt %, more preferably less than 30 wt %, more preferably still less than 20 wt %, and especially preferably less than 10 wt %, for example less than 5 wt %, or indeed less than 1 wt %, based on the weight of the coating material. The coating may also contain no pigments and/or fillers.

Alternatively or additionally, according to certain embodiments, the coating material may comprise additives which influence the optical and/or technical properties. The additives, for example, may act as lubricity assistants and/or defoamers. There is no particular restriction on these additives, which may be, for example, waxes and/or surface-active substances (e.g. silicones, surfactants). According to certain embodiments, the amount in which such additives are added may be 0-10 wt %, preferably 0-5 wt %, more preferably 0-2 wt %, based on the weight of the coating material. The coating may also not contain any further additives. Alternatively or additionally, according to certain embodiments, the coating material may comprise plasticizers and/or humectants. There is no particular restriction on these plasticizers and/or humectants, which may, for example, comprise glycerol, urea and/or triacetin. There is no particular restriction on the amount in which such plasticizers and/or humectants are added, the amount according to certain embodiments possibly being 0-30 wt %, preferably 0-15 wt %, more preferably 0-10 wt %, based on the weight of the coating material.

According to certain embodiments, the coating has a dry film thickness of 0.1 μm to 10 μm, preferably of 0.5 to 5 μm, more preferably of 1 μm to 3 μm. According to certain embodiments, this thickness is obtained on only one side, on two or more sides or in each case on one side of the foil after drying, in a stream of gas or in a drying oven, for example. The dry film thickness in this context may be determined, for example, by means of spectral reflection, using an instrument from Filmetrics, for example. Although in practice it is not possible to apply arbitrarily thin layers, an improved gloss effect may be achieved with relatively thin layers.

The amount of metal pigments, more particularly aluminium pigments, may according to certain embodiments be 0.01-10 wt %, preferably 0.01-5 wt %, more preferably 0.01-1 wt % and especially preferably 0.01-0.5 wt %, based on the weight of the foil. This increases the resistance of the glitters to water in particular. According to certain embodiments, the glitters are at least twice as large, based on a surface to be coated on one side of the glitters, as the metal pigments.

Furthermore, the coated glitter of the invention may also comprise one or more further layers, examples being layers which impart colour and/or effect or else layers of other kinds, examples being layers for improving the UV stability, abrasion resistance and/or water resistance, these layers being of kinds known to the skilled person and being able to be suitably applied by the skilled person, from the gas phase and/or from liquid/solution, for example. There is no particular restriction on these coatings, which may be produced, for example, on the basis of cellulose or modified cellulose, preferably based on cellulose nitrate, cellulose acetate butyrate, cellulose acetate propionate and/or cellulose acetate, and/or based on polyvinyl alcohol and/or shellack and/or polyurethane and/or polyvinylpyrrolidone, and/or based on natural resins, preferably dammar and/or kopal and/or sandarak gum and/or gum Arabic and/or zein. According to certain embodiments, the stated binders may have a fraction of 10-99.9 wt %, based on the weight of the coating material.

According to certain embodiments, the foil of the glitters of the invention has a thickness of 5 μm or more, preferably of more than 10 μm, more preferably of more than 13 μm, and/or a thickness of 50 μm or less, for example less than 40 μm, preferably 36 μm or less.

In the coated glitter of the invention the foil may be coloured or uncoloured, but according to certain embodiments is uncoloured. In certain embodiments, moreover, a colour-imparting and/or effect imparting or else other kind of layer may be applied to the coating comprising metal pigments, more particularly aluminium pigments, or to the foil. The coating comprising metal pigments, more particularly aluminium pigments, is preferably applied directly to the foil, more particularly on all sides of the foil, although a coating on one side already increases the stability. If the coating comprising metal pigments, more particularly aluminium pigments, is not applied on all sides of the foil, it is also possible for the foil to be coated—at least partially—with a colour-imparting and/or effect-imparting or else other kind of layer.

According to certain embodiments, the foil in a glitter of the invention may also be produced with a hologram imprint, of the kind known for conventional glitter, for example, from U.S. Pat. No. 5,810,957 or EP 2163381 A, both documents being referenced with regard to the hologram imprint. It is also possible, surprisingly, to realize the hologram imprint on foils coated with metal pigments, more particularly aluminium pigments. For production in this case it is possible to use common methods, such as those known as soft embossing and hard embossing.

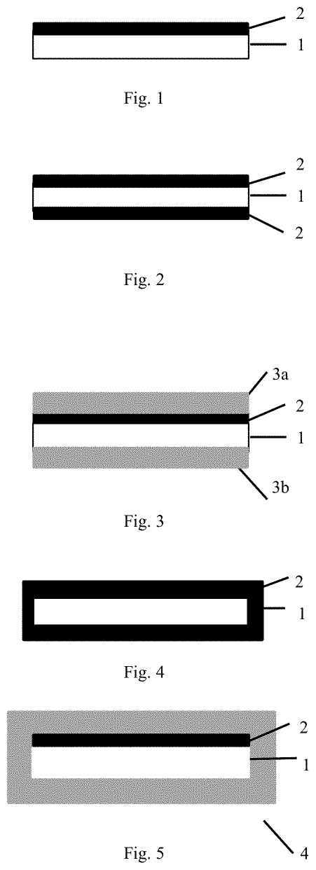

Illustrative coated glitters of the invention are elucidated in more detail below, on the basis of illustrative embodiments, which are shown in FIGS. 1 to 5. The figures here show, schematically, sectional views through glitters having a construction in accordance with the invention.

According to a first illustrative embodiment, which is shown in FIG. 1, for example, particles 1 consisting of a foil comprising cellulose and/or a derivative thereof, e.g. comprising cellulose and/or cellulose acetate, are coated on one side with a coating comprising metal pigments, for example with a cellulose acetate layer 2 which contains aluminium pigments. The coating comprising metal pigments, for example composed of cellulose acetate and aluminium pigments, is applied, for example, from a liquid phase to a film comprising cellulose and/or a derivative thereof, which for example may also be transparent, by means of a gravure printing process, for example. The glitter particles may then be produced from the coated foil by cutting.

According to a further embodiment, which is shown in FIG. 2, particles 1 consisting, for example, of a foil comprising cellulose and/or a derivative thereof, e.g. comprising cellulose and/or cellulose acetate, may be coated on two opposite sides, with a cellulose acetate layer 2 containing aluminium pigments, for example. The coating of cellulose acetate and aluminium pigments may be applied from a liquid phase to a foil comprising cellulose and/or a derivative thereof, e.g. comprising cellulose and/or cellulose acetate, by means of gravure printing processes, for example, in which case both sides of the foil can be printed. The glitter particles may be produced from the coated foil by cutting.

Furthermore, the particles 1 consisting of a foil comprising cellulose and/or a derivative thereof, e.g. comprising cellulose and/or cellulose acetate, and coated on one side with a coating containing aluminium pigments, a cellulose acetate layer 2, for example, may additionally, in a further embodiment, as shown in FIG. 3, be coated on two opposite sides uniformly with further layers 3a, 3b of cellulose acetate, for example. These layers may be applied by printing, for example, like the cellulose acetate layer 2 which comprises aluminium pigments.

Alternatively, in a further embodiment, as shown in FIG. 4, particles 1 consisting of a foil comprising cellulose and/or a derivative thereof, e.g. comprising cellulose and/or cellulose acetate, may be coated on all sides uniformly with a coating comprising metal pigments, with a cellulose acetate layer 2 containing aluminium pigments, for example. Such coating may be accomplished, for example, by coating in a liquid or gas phase.

Furthermore, the particles 1 consisting of a foil comprising cellulose and/or a derivative thereof, e.g. comprising cellulose and/or cellulose acetate, and coated on one side with a coating comprising metal pigments, with a cellulose acetate layer 2 containing aluminium pigments, for example, may be coated additionally, in a further embodiment, as shown in FIG. 5, uniformly on all sides with a further layer 4 of cellulose acetate, for example. This further layer may be applied, for example, in a fluidized bed or in another way, including, for example, after the cutting with the particles coated with layer 2.

In FIGS. 1 to 5, according to alternative configurations, the foil may also be coloured. In FIGS. 3 to 5, moreover, the layers 3a, 3b and/or 4, according to alternative configurations, may also be coloured.

A further aspect of the present invention is directed to the use of coated glitters of the invention in a cosmetic product. There are no particular restrictions here on the cosmetic product. Cosmetic products here encompass, for example, pastes, salves, creams, emulsions, solutions, lipstick, lip gloss, mascara, mousse, eye shadow, eye liner, powder, pressed powder, loose glitter powder, nail varnish, soaps, shampoo, sun protection products, lotions, aerosol sprays, etc., which may comprise the glitters in usual amounts in the formulations.

Also disclosed is a cosmetic product which comprises coated glitters of the invention. There are no particular restrictions here on the cosmetic product, and the product may be, for example, a paste, a salve, a cream, an emulsion, a solution, a lipstick, lip gloss, mascara, mousse, eye shadow, eye liner, powder, pressed powder, loose glitter powder, nail varnish, soap, shampoo, sun protection product, a lotion, an aerosol spray, etc., where the glitters may be present in the cosmetic product in customary amounts, for example between 0.01 and 75 wt %, e.g. between 1 and 10 wt %, based on the cosmetic product, or, indeed, up to 100 wt % in the case of powder and loose glitter powder. In the cosmetic products the customary constituents may be present as well, such as carriers, fillers, oils, waxes, greases, emulsifiers, antioxidants, film formers, odorants and/or flavours, stabilizers, solvents, surfactants, preservatives, thickeners, rheological additives, dyes, vitamins, buffer substances, active cosmetic ingredients, skin-active substances, e.g. skincare substances, UV filters, etc., all of which are not subject to particular restriction. The cosmetic products may for example be hydrophilic, hydrophobic and/or lipophilic. Corresponding constituents are known, for example, from DE 102005055576 Al, which is referenced illustratively in relation to cosmetic formulations for the production of cosmetic products.

The glitters of the invention are compatible with a multiplicity of typical formulations from the sectors of paints, varnishes, adhesives or moulding and modelling compositions. Disclosed accordingly is the use of the coated glitters of the invention in coating materials, including coating systems, for example, preferably from the sector of paints, varnishes or printing inks, which here are not subject to particular restriction, it being possible for the glitters to be present in customary amounts.

For use in printing inks, for gravure printing or flexographic printing, for example, there are a multiplicity of water-thinnable or water-based printing inks and overprint varnishes that are suitable for example, as sold for example by the companies Marabu, Pröll, Hartmann, Siegwerk, Rotoflex, GSB-Wahl and/or Coates Screen, and in which glitters of the invention may be used. The coating materials in question may be dried and/or cured, for example, thermally or by radiation curing.

For the use of the coated glitters of the invention in paints and varnishes there are a multiplicity of suitable water-thinnable or water-based formulations, which are not subject to particular restriction. There is no particular restriction on the application of the coating materials, which is typically accomplished by brushing, rolling, by a spray process, in the form of aerosol spray, or by another suitable process. Likewise suitable are finger paints, modelling paints or artist's colours.

Further disclosed is a coating material which comprises the coated glitter of the invention. There is no particular restriction here on the amount of the glitters in the coating material, which may comprise customary amounts, depending on the particular coating material. In coating materials the glitters of the invention are used, for example, in concentrations of 0.1 wt % to 70 wt %, preferably in concentrations of 0.5 wt % to 50 wt %, more preferably in concentrations of 0.5 wt % to 10 wt %, based on the coating material. Examples of coating materials contemplated include paints or varnishes, including printing inks, and also, for example, finger paints, modelling paints or artist's colours.

Also disclosed is the use of the coated glitters of the invention in an adhesive, which is not subject to particular restriction. For use in, for example, water-thinnable or water-based adhesives, such as modelling adhesive, all-purpose adhesive or paper adhesive, for example, there is no particular restriction here on the systems that are suitable.

Also disclosed is an adhesive comprising the coated glitter of the invention. There is no particular restriction here on the amount of the glitters in the adhesive, which may comprise customary amounts, depending on the particular adhesive. In adhesives the glitters of the invention are used, for example, in concentrations of 0.1 wt % to 70 wt %, preferably in concentrations of 0.5 wt % to 50 wt %, more preferably in concentrations of 0.5 wt % to 10 wt %, based on the adhesive. Examples of adhesives contemplated include water-thinnable or water-based adhesives, such as modelling adhesive, all-purpose adhesive or paper adhesive, for example.

The coated glitters of the invention are also suitable for use in modelling compositions and moulding compositions, examples being those containing water, such as play dough and modelling clay or papier mâché, and which are not subject to particular restriction.

Further disclosed is a modelling or moulding composition which comprises the coated glitter of the invention. There are no particular restrictions on the modelling or moulding composition, which comprises, for example, water-containing modelling compositions and moulding compositions, such as, for example, play dough and modelling clay and also papier mâché. There are no particular restrictions on the amount of the glitters in the modelling or moulding composition, and the amount may comprise customary amounts, depending on the particular modelling or moulding composition. In modelling or moulding compositions, the coated glitters of the invention are used for example in concentrations of 0.1 wt % to 60 wt %, preferably in concentrations of 0.5 wt % to 20 wt %, more preferably in concentrations of 0.5 wt % to 10 wt %, based on the modelling or moulding composition.

Further disclosed is a method for producing a coated glitter, comprising: providing a foil, where the foil comprises cellulose and/or a derivative thereof; producing a coating comprising metal pigments, more particularly aluminium pigments, on at least one side of the foil, including, for example, on two or more sides of the foil or on the entire foil; and producing the coated glitter; or providing a foil, where the foil comprises cellulose and/or a derivative thereof; cutting the foil into suitably sized uncoated glitter particles; and coating the uncoated glitter particles with metal pigments, more particularly aluminium pigments, for example on at least one side of the foil, including for example on two or more sides of the foil or on the entire foil.

The method of the invention is used in particular to produce coated glitters of the invention. Accordingly, aspects disclosed in connection with the coated glitter of the invention may also find corresponding application in the context of the method of the invention, in relation, for example, to the nature of the foil, coating, etc., or in relation to any further layers, which where appropriate may be applied suitably further, and vice versa. Individual embodiments as well, already described in connection with the coated glitter, may also find application correspondingly in the method of the invention, in relation, for example, to weight fractions of particular substances, etc., and vice versa.

The method for producing the glitters of the invention, furthermore, is not subject to particular restriction. A foil comprising a coating with metal pigments, more particularly aluminium pigments, may here, for example, be suitably cut into suitably sized particles and applied optionally in a customary way with polymers—such as those described illustratively above, pigments, etc. There are no particular restrictions on the application of various coatings, which may take place, for example, to the foil (in the form of a roll product) and/or to the particles, in a fluidized bed, for example.

In the method of the invention there are no particular restrictions on the provision of the foil.

A coating may be produced in a suitable way, for example by application of the metal pigments, optionally with a coating material and/or optionally further components, as described above in connection with the glitter of the invention, to at least one side of the foil. Application may take place in any desired way, for example from the gas phase and/or liquid phase—in which case the liquid phase may also comprise solids, for example from suspension, etc. According to certain embodiments, coating takes place from a liquid phase, using a suspension, a dispersion, etc., for example. The coating in this case may take place, according to certain embodiments, on one side, on two opposite sides, on the entire foil, or in another way. The metal pigments, more particularly aluminium pigments, may be applied to the foil preferably from a liquid phase. The metal pigments, more particularly aluminium pigments, may be present, for example, as a dispersion in a solvent or solvent mixture. The metal pigments, more particularly aluminium pigments, may alternatively be part of a liquid coating material which may comprise polymeric binders, additives, fillers or solvents. A liquid phase comprising the metal pigments, more particularly aluminium pigments, may be applied by means of coating methods known to the skilled person, such as gravure printing, flexographic printing or screen printing, for example, but its application is not confined to a particular method.

There are no particular restrictions on suitable metal pigments, more particularly aluminium pigments, which are produced and sold, for example, by Eckart GmbH (brand names e.g. Rotovario, Stapa, Silveroto, Metalure, Standart, Visionaire) or by Carl Schlenk AG (brand names e.g. Alushine, Alustar, Aquamet, Decomet, Alegrace).

Production of the coated glitter comprises, for example, the cutting of the coated foil, this cutting not being subject to particular restriction.

In the alternative method, after the cutting of the foil, the coating of the uncoated glitter particles with metal pigments, more particularly aluminium pigments, is not subject to particular restriction, and may be accomplished, for example, from a liquid phase, as described for example in connection with the production of a coating, or else by immersion, etc., or else from the liquid-gas phase, by coating with a spray, aerosol, etc., for example, or in another suitable way.

According to certain embodiments, coating takes place from a liquid phase, using a suspension, a dispersion, etc., for example. According to certain embodiments, coating takes place with metal pigments, more particularly aluminium pigments, from a liquid phase, more particularly on the foil.

The above embodiments, configurations and developments may be combined with one another as desired, where such combination is rational. Further possible configurations, developments and implementations of the invention also encompass combinations, not stated explicitly, of features of the invention that are described above or below with reference to the exemplary embodiments. In particular, the skilled person will also add individual aspects as improvements or supplementations to the respective basic form of the present invention.

The invention is elucidated further in detail below with reference to various examples thereof. The invention, however, is not confined to these examples.

### EXAMPLES

### Inventive Example 1: Coating in a Gravure Printing Process

Glitters of the invention according to FIG. 1 were produced by coating of a foil containing 87 wt % cellulose, 10 wt % glycerol and 3 wt % urea, with a thickness of 17 μm to 23 μm, with aluminium pigments by printing in a gravure printing process on one side and subsequent cutting to a size of 200 μm+/−20 μm. The coating material is produced by dissolving 100 g of cellulose acetate in 1000 g of methyl ethyl ketone. Incorporated into this solution with stirring are 100 g of aluminium pigment (10% dispersion in ethyl acetate). Using an engraved roll (70 LPI×127 μm), the coating material is applied to the foil with 2.7 wt % of pigment, based on the foil, and is subsequently dried at 120° C. The result was a coating on which the aluminium pigments were clearly visible in the form of dot-like regions.

### Comparative Example 1

For comparison, a comparative glitter composed of the same cellulose-containing foil, coated in a vacuum process with aluminium (0.5 wt % under vacuum) and produced by corresponding methods with the same size as in Inventive Example 1, was used. Following the vacuum coating, a cellulose acetate coating was applied as in Inventive Example 1, but without aluminium pigment. The result in this case was a uniform coating.

### Test Example 1: Glitter Stability

Using the two glitters (glitter of Inventive Example 1 and comparative glitter of Comparative Example 1), the resistance to water was tested by introduction into fully demineralized water (FD water) for 24 h at 23° C. The respective glitter is introduced in a test tube and covered with a layer of FD water. Assessment takes place visually. The results are shown in Table 1.

TABLE 1
Water resistance of the glitters from Inventive
Example 1 and Comparative Example 1

| | | Cellulose glitter with vacuum metallization |
|---|---|---|
| | | SiLiglam PURE BIO SPARKLE |
| | Glitter of the invention | 1610-50-2hex, from Sigmund Lindner |
| | (Inventive Example 1) | GmbH (Comparative Example 1) |
| FD water (24 h at 23° C.) | No visible alteration | Loss of lustre |

The resistance to 25% ammonia solution in water was also tested using the two materials. The respective glitter is placed in a test tube and covered with a layer of ammonia solution. Assessment takes place visually. The results are shown in Table 2.

TABLE 2
Ammonia resistance of the glitters from
Inventive Example 1 and Comparative Example 1

| | | Cellulose glitter with vacuum metallization |
|---|---|---|
| | | SiLiglam PURE BIO SPARKLE |
| | Glitter of the invention | 1610-50-2hex, from Sigmund Lindner |
| | (Inventive Example 1) | GmbH (Comparative Example 1) |
| 25% ammonia solution | No visible alteration after | Glitter becomes transparent after 5 minutes |
| | 30 minutes | |

Inventive Example 2a: Coating in a Fluidized Bed Process on Cellulose Particles Transparent glitter particles (SiLiglam PURE BIO SPARKLE 1610-11-2hex, particle size 0.2 mm, from Sigmund Lindner GmbH), produced from a cellulose foil having a thickness of 17 μm to 23 μm, are coated with aluminium pigments by a fluidized bed process. To produce the coating material, 1000 g of shellack solution (50% in ethanol) are introduced. Incorporated into this solution with stirring are 100 g of aluminium pigment dispersion (10% dispersion in ethyl acetate).

3 kg of transparent glitter particles are charged to the materials container of a fluidized bed unit (ProCell Lab, from Glatt) and fluidized using process air, preheated to 80° C., with an air throughput of 35 m³/h, and a fluidized bed is formed. In what is referred to as a bottom spray process, the coating solution is sprayed into the fluidized bed at a rate of 10 ml/min. The glitter particles coated in this way are withdrawn from the machine and cooled to room temperature. The glitter particles thus produced correspond to the schematic construction shown in FIG. 4.

### Inventive Example 2b: Coating in a Fluidized Bed Process on Cellulose Acetate Particles

Analogous to Inventive Example 2b, glitters based on the cellulose derivative cellulose acetate are produced. Transparent glitter particles (SiLiglam PURE NATURE 1710-11-2hex, particle size 0.2 mm, from Sigmund Lindner GmbH), produced from a cellulose acetate foil having a thickness of 10 μm to 15 μm, are coated with aluminium pigments by a fluidized bed process. To produce the coating material, 1000 g of shellack solution (50% in ethanol) are introduced. Incorporated into this solution with stirring are 100 g of aluminium pigment dispersion (10% dispersion in ethyl acetate).

3 kg of transparent glitter particles are charged to the materials container of a fluidized bed unit (ProCell Lab, from Glatt) and fluidized using process air, preheated to 80° C., with an air throughput of 35 m³/h, and a fluidized bed is formed. In what is referred to as a bottom spray process, the coating solution is sprayed into the fluidized bed at a rate of 10 ml/min. The glitter particles coated in this way are withdrawn from the machine and cooled to room temperature. The glitter particles thus produced correspond to the schematic construction shown in FIG. 4.

### Comparative Example 2

For comparison, glitter composed of cellulose-containing foil from Inventive Example 1, coated with aluminium in a vacuum process and produced by corresponding processes with the same size, was used. Following the application of the aluminium, a shellack solution as in Inventive Example 2, but without aluminium, was applied. Furthermore, a glitter based on the cellulose acetate foil from Inventive Example 2b was coated accordingly with aluminium and produced with the same size, and a shellack solution was applied. Also subjected to the stability test as a reference was SiLiglam PURE BIO SPARKLE 1610-11-2hex from Inventive Example 2, which is transparent.

### Test Example 2: Glitter Stability

The resistance to water-based hair gel (Balea MEN, Styling Gel maximum power, from dm) was tested using the materials from Inventive Example 2a, Inventive Example 2b, and Comparative Example 2. The glitter is introduced into respective test tubes and covered with a layer of hair gel. The samples are stored in a laboratory oven at 50° C. Assessment is made visually. After oven storage for 4 weeks, there is no visible alteration to the glitter of the invention from Inventive Example 2a and the glitter of the invention from Inventive Example 2b. In the case of the comparative glitter from Comparative Example 2 (both with the cellulose and the cellulose acetate foil), complete decolorization has occurred, attributable to the fact that the aluminium layer applied by the vacuum process has passed into solution. In the case of the SiLiglam PURE BIO SPARKLE 1610-11-2hex glitter, there was no visual alteration, and the glitter was transparent from start to finish.

### Inventive Example 3: Coating in a Fluidized Bed Process

Transparent glitter particles (SiLiglam PURE BIO SPARKLE 1604-11-2hex, particle size 1.0 mm, from Sigmund Lindner GmbH), produced from a cellulose foil having a thickness of 17 μm to 23 μm, are coated with aluminium pigments by a fluidized bed process. To produce the coating material, 250 g of polyurethane dispersion are introduced. Incorporated into this dispersion with stirring are 1150 g of aluminium pigment dispersion (10% dispersion in isopropyl alcohol).

3 kg of transparent glitter particles are charged to the materials container of a fluidized bed unit (ProCell Lab, from Glatt) and fluidized using process air, preheated to 80° C., with an air throughput of 35 m³/h, and a fluidized bed is formed. In what is referred to as a bottom spray process, the coating solution is sprayed into the fluidized bed at a rate of 10 ml/min. The glitter particles coated in this way are withdrawn from the machine and cooled to room temperature.

### Comparative Example 3

For comparison, a commercially available glitter (from Ronald Britton, BioGlitter Sparkle, silver, 0.040) was used, based on regenerated cellulose, in the same particle size as the glitter of the invention.

### Test Example 3: Glitter Stability

The dimensional stability in water was tested using the two materials from Inventive Example 3 and Comparative Example 3, by introducing them into fully demineralized water (FD water) at 50° C. for 30 minutes. The glitter is introduced into respective test tubes and covered with a layer of FD water. Assessment is made visually. The results are shown in Table 3.

TABLE 3
Dimensional stability of the glitters from
Inventive Example 3 and Comparative Example 3

| | | Cellulose glitter with vacuum |
|---|---|---|
| | | metallization, |
| | Glitter of the invention | BioGlitter Sparkle, silver, 0.040, from |
| | (Inventive Example 3) | Ronald Britton (Comparative Example 3) |
| FD water (30 min at | No visible alteration | Visible deformation and curling of the glitter |
| 50° C.) | | particles |

### Example 4: Application Examples of Glitters of the Invention

### Product 1: Hair Wax

| Phase | Ingredient | % w/w |
|---|---|---|
| A | Water | 45.5 |
| | Propylene glycol | 4.0 |
| | PVP (Polyvinylpyrrolidone) | 6.0 |
| | Glitter of Inventive Example 2 | 2.0 |
| B | Cera alba, Ceteareth-25 | 38 |
| | Octyl dodecanol | 4 |
| | Phenoxyethanol (and) ethylhexyl | 0.5 |
| | glycerol | |
| | Total | 100.0 |

### Production:

Mix phases A and B each separately and stir them until a homogeneous solution is formed. Heat phases A and B separately from one another to 90-95° C. Mix the two phases with stirring and leave the mixture to cool.

### Storage Stability: >4 Weeks at 50° C.

### Product 2: Modelling Adhesive

| Ingredient | % w/w |
|---|---|
| ÖkoNorm Pro Coll paper glue | 98.5 |
| (glucose-saccharide mixture, sucrose, | |
| water, glycerol, phenoxyethanol) | |
| Glitter from Inventive Example 2 | 1.5 |
| Total | 100.0 |

### Production:

Add glitter to the adhesive with stirring and homogenize.

### Storage Stability: >4 Weeks at 50° C.

### Product 3: Gloss Varnish, Water-Thinnable

| Ingredient | % w/w |
|---|---|
| Alberdingk AC 2742, Alberdingk-Boley | 71.6 |
| 2-Butoxyethanol | 5 |
| Water | 19 |
| Byk 333, Byk | 3 |
| Byk 349, Byk | 0.2 |
| DSX 1514, BASF | 0.2 |
| Glitter from Inventive Example 2 | 1.0 |
| Total | 100.0 |

### Production:

Mix ingredients with stirring

### Storage Stability: >4 Weeks at 50° C.

### Product 4: Play Dough

| Ingredient | % w/w |
|---|---|
| Kinder Soft Knete yellow dough, Feuchtmann | 99 |
| Glitter from Inventive Example 2 | 1 |
| Total | 100.0 |

### Production:

Add glitter to the play dough and homogenize.

### Storage Stability: >4 Weeks at 50° C.

1. A coated glitter comprising a foil which has a coating comprising metal pigments, more particularly aluminum pigments, wherein the foil comprises cellulose and/or a derivative thereof.

2. The coated glitter according to claim 1, wherein the foil comprises at least 60 wt % of cellulose and/or a derivative thereof, based on the weight of the foil.

3. The coated glitter according to claim 1, wherein the foil consists substantially of cellulose and/or a derivative thereof.

4. The coated glitter according to claim 1, wherein the coating has a dry film thickness of 0.1 μm to 10 μm.

5. The coated glitter according to claim 1, wherein the glitter contains 0.01-10 wt % of metal pigments, more particularly aluminum pigments, based on the weight of the foil.

6. Use of a coated glitter according to claim 1 in a cosmetic product, in a coating material, in an adhesive, or in a modelling and/or moulding composition.

7. A cosmetic product comprising a coated glitter according to claim 1.

8. A coating material comprising a coated glitter according to claim 1.

9. An adhesive comprising a coated glitter according to any of claim 1.

10. A modelling and/or moulding composition comprising a coated glitter according to claim 1.

11. A method for producing a coated glitter, comprising:
providing a foil, where the foil comprises cellulose and/or a derivative thereof;
producing a coating comprising metal pigments, more particularly aluminum pigments, on at least one side of the foil; and
producing the coated glitter; or
providing a foil, where the foil comprises cellulose and/or a derivative thereof;
cutting the foil into suitably sized uncoated glitter particles; and
coating the uncoated glitter particles with metal pigments, more particularly aluminum pigments.

12. The method according to claim 11, wherein the coating with metal pigments, more particularly aluminum pigments, takes place from liquid phase, more particularly on the foil.
